(19) United States
(12) Patent Application Publication
(10) Pub. No.: US 2002/0035100 A1
(43) Pub. Date: Mar. 21, 2002

(54) Alkyl ether modified polycyclic compounds having a terminal phenol and uses for protection of cells

(76) Inventors: Laszlo Prokai, Gainesville, FL (US); James W. Simpkins, Fort Worth, TX (US)

Correspondence Address: BROMBERG & SUNSTEIN LLP, 125 SUMMER STREET, BOSTON, MA, 02110-1618, US

(21) Appl. No.: 09/893,324

(22) Filed: Jun. 27, 2001

Related U.S. Application Data
(60) Provisional application No. 60/214,077, filed on Jun. 27, 2000.

(51) Int. Cl. A61K 31/56; C07J 1/00

(52) U.S. Cl. 514/182; 552/625

(57) ABSTRACT

Methods and compositions are provided for achieving a cytoprotective effect by selecting a polycyclic compound with a phenol group at one end of the molecule and a carbon ring at the other such that an alkyl ether functional group in which the alkyl group has a formula CₙH₂ₙ₊₁ (where n is at least 3 and less than 20) is positioned on the carbon ring. The compound may be used to achieve a cytoprotective effect in cells and to retard the development of a degenerative condition in a subject suffering from a disease, trauma or aging.

### CROSS REFERENCE TO RELATED APPLICATIONS

This application gains priority from the provisional application filed Jun. 27, 2000 herein incorporated by reference.

### GOVERNMENT RIGHTS

This patent was created with support from the National Institute on Aging under grant number POI 10485. The U.S. Government has certain rights to the invention.

### TECHNICAL FIELD AND BACKGROUND ART

The present invention relates to methods and compositions to achieve a cytoprotective effect concerning a polycyclic compound with a phenol group at a first end and a carbon ring at a second end in which the hydroxy group on the carbon ring has been substituted by an alkyl ether group.

The naturally occurring hormone 17β-estradiol plays a pivotal role in sexual reproduction in humans and other mammals. It is believed that this estrogenic activity is orchestrated through the binding of estrogen receptors on the surface of target cells (Gridley et al. (1998) Vol. 54, pp. 874-880). Estrogen compounds including 17β-estradiol have also been shown to have neuroprotective activity (U.S. Pat. No. 5,554,601). More generally, cytoprotective activity has been demonstrated for estrogen compounds that have little or no estrogenic activity and in addition have low or negligable binding affinity for the estrogen receptor (U.S. Pat. No. 5,843,934). An important functional group in these molecules that determine cytoprotection is the presence of a terminal phenolic group. This observation led to the realization that polycyclic compounds had neuroprotective activity contingent on the presence of a terminal phenol group. (U.S. Pat. Nos. 5,859,001, 6,197,833) (Bishop et al. (1994) Mol. Cell. Neurosci, Vol. 5, pp. 303-308; Green et al. (1997) J. Steroid Biochem. Mol. Biol., Vol. 63, pp. 229-235).

The above described cytoprotective activity has numerous uses in protecting cells in vivo and in vitro from degeneration that may occur through disease, trauma or aging. Treatment based on cytoprotection can lead to the slowing of progression of degeneration and postpone the onset of symptoms associated with degeneration. It is desirable therefore, to identify improvements in cytoprotective compounds that might enhance their bioactivity.

### SUMMARY OF THE INVENTION

A first embodiment of the invention provides a cytoprotective compound that includes a polycyclic compound optionally having two, three or four carbon rings, the compound also having a first end and a second end wherein a phenol group is located at the first end and a terminal carbon ring is located at the second end, the terminal carbon ring having an alkyl ether functional group, the alkyl portion of which having a formula CₙH₂ₙ₊₂ wherein n is at least 3 and less than 20.

In additional embodiments, the carbon ring at the second end is a D ring in a four ring compound which may be an estrogen. The four ring estrogen compound may include an alkyl ether group in an alpha or beta orientation. Moreover the alkyl ether functional group can include any of a long chain saturated alkyl, a long chain unsaturated alkyl, or a cycloalkyl group. In specific embodiments, the cytoprotective compound may be a 17-butoxyestra 1,3,5(10)triene-3-ol, 17-pentoxyestra 1,3,5(10)triene-3-ol a 17-hexoxyestra 1,3,5(10) triene-3-ol, a 17septoxyestra 1,3,5(10)triene-3-ol, or a 17-octyloxyestra 1,3,5(10)triene-3-ol.

In a second embodiment of the invention, the cytoprotective compound includes an estrogen compound having a terminal phenol group at a first end of the compound and a carbon ring at a second end of the compound, the carbon ring at the second end having an alkyl ether functional group wherein the alkyl group has a formula CₙH₂ₙ₊₂ wherein n is at least 3 and less than 20.

In a third embodiment of the invention, a pharmaceutical formulation is provided that includes a cytoprotection effective dose of a polycyclic compound having a phenolic ring at a first terminal position, optionally any of one, two or three additional ring structures and an alkyl ether functional group on a carbon ring in a second terminal position.

In a fourth embodiment of the invention, a method is provided for retarding the development of a degenerative condition associated with a population of cells in a subject, that includes administering to the subject predisposed to the degenerative condition, an effective amount of a polycyclic phenolic compound in a physiologically acceptable formulation, the polycyclic phenolic compound having a phenol located at a first terminal position, optionally any of one, two or three additional ring structures; the compound having an alkyl ester located on a carbon ring at a second terminal position, the compound retarding the development of the degenerative condition. The method may utilize any of the alkyl ether compounds described herein including four ring compounds with an alkyl ether on carbon 17 of the D ring in an alpha or beta orientation and may further include enantiomers, diastomers, salts, derivatives and analogs.

The population of cells or tissues may be selected from stem cells, blood cells, epithelial cells, stromal cells including connective tissue cells, neuronal cells, muscle tissue cells, endocrine tissue cells, whole organ cells, bone cells, eye cells, skin cells, reproductive tract cells and urinary tract cells. The degenerative condition may include cardiac, eye, bone, neurodegenerative or ischemic degeneration.

In a fifth embodiment of the invention, a method is provided for synthesizing an estrogen compound having a phenolic A ring and an alkyl ether functional group on carbon 17, that includes: protecting —OH on the phenolic A ring; alkylating the 17-OH with an alkylating agent in the presence of a strong base; removing the protecting group from —OH on the phenolic A ring; and purifying the 17-alkyl ether estrogen compound. Moreover, the —OH may be on the carbon 3-position and the 17-OH may be in an alpha or beta position. The alkylating agent may be selected from a group consisting of an alkyl halide, a dialkyl sulfate and an alkyl tosylate. The phenolic-OH may be treated with a base resistant protecting group such as tert-butyl, methoxymethyl and 9-anthrylmethyl. The protecting group may be removable by acid hydrolysis, catalytic hydrogenolysis where the hydrogenolysis may include CF₃COOH or by catalytic transfer hydrogenation which may use ammonium formate. The strong base of the method may include sodium hydride.

In a sixth embodiment of the invention, a method is provided for treating a subject having a degenerative disorder, comprising: obtaining at least one 17-O-alkyl ether of estrogen in a pharmaceutical formulation; and administering an effective dose of the 17-O-alkyl ether of estrogen to the subject so as to treat the degenerative disorder.

In a seventh embodiment of the invention, a method is provided for conferring cytoprotection of a population of cells, that includes providing an 17β-O-alkyl ether of an estrogen compound; and administering the compound in an effective dose to the population of cells so as to confer cytoprotection on the population of cells. All embodiments directed to methods include the use of any of the alkyl ether compounds described herein

### BRIEF DESCRIPTION OF THE DRAWINGS

The foregoing features of the invention will be more readily understood by reference to the following detailed description, taken with reference to the accompanying drawings, in which:

FIG. 1 shows the structure of the alkyl ether of estradiol.

FIG. 2 shows the synthesis of 17-alkyl ether of estradiol.

FIG. 3 is an ORTEP plot of the X-ray crystal structure of 17-O-butylated 17β-estradiol (4d). Thermal ellipsoids are shown at the 30% probability level.

FIG. 4 shows a graphical representation of cell viability, where the cells are HT-22 cell cultures after glutamate exposure (20 mM) (a) following treatment with estradiol and its 17β-alkyl ethers (4a-4f), and 3-butyl estradiol (5b as a typical representative of the 3-alkyl ethers). Statistically significant differences between groups were tested by analysis of variance (ANOVA) followed by post hoc Tukey test: * significant increase (p<0.05) vs vehicle control, ** significant increase (p<0.05) vs vehicle control, but decrease compared to 10 μM estradiol (1), *** increase (p<0.05) vs vehicle control, and statistically significant increase compared to 10 μM estradiol (1).

### DETAILED DESCRIPTION OF SPECIFIC EMBODIMENTS

As used in this description and the accompanying claims, the following terms shall have the meanings indicated, unless the context otherwise requires:

“Estrogen compound” is defined here and in the claims as any of the structures described in the 11^{th} edition of “Steroids” from Steraloids, Inc. Wilton, N.H., here incorporated by reference. Included in this definition are non-steroidal estrogens described in the aforementioned reference. Other estrogen compounds included in this definition are cyclopenantophenanthrene compounds, estrogen derivatives, estrogen metabolites and estrogen precursors as well as those molecules capable of binding cell associated estrogen receptor as well as other molecules where the result of binding specifically triggers a characterized estrogen effect. Assumed as included in this definition but more explicitly stated, are isomers, diasteromers and enantiomers of the aforementioned, as well as mixtures of more than one estrogen.

In an embodiment of the invention, “cytoprotective effect” is a measurable positive effect on the survival of cells that would otherwise die without an intervention.

“Treatment” of a disorder in a patient with a cytoprotective compound may be characterized as, but is not limited to, a slowing of progression of a disorder and optionally slowing of the development of symptoms than would otherwise occur in the absence of the compound.

“Alkyl ether functional group on the carbon ring at the second end” includes locating the alkyl ether functional group on any available carbon in the ring for example, carbon-15, -16 or -17. “terminal phenol group” includes a carbon ring with an OH— group on any of carbons 2, 3 or 4.

“Alkyl ether functional group on carbon 17 of the D ring ” refers unless specified otherwise to 17β-, 17α-, enantiomers of the four ring compound, salts, derivatives and analogs thereof. Similarly, a 17-alkylestra-1,3,5(10) triene-3-ol refers to any of the 17-α or 17-β diesteromer, and the enantiomers of the compound, salts, derivatives and analogs thereof.

“17-” refers to 17β- or 17α-.

We have synthesized novel modifications of known compounds that have improved cytoprotective activity when compared with the unmodified forms. The novel compounds are polycyclic compounds with a terminal phenol group that have been modified in such a way as to increase the lipophilicity of the compounds for improved uptake by target cells thereby improving the cytoprotective effect of the compounds while maintaining the terminal phenol group. Polycyclic compounds with a terminal phenol group prior to modification with an alkyl ether as described below include those compounds listed in U.S. Pat. No. 6,197,833 herein incorporated by reference. Embodiments of the invention include compounds with significantly less feminizing activity compared with 17β-estradiol and include compounds that do not readily bind the estrogen receptor (Table 10). Accordingly, modifications include the addition of an alkyl ether on carbon 17 of the molecule, where the alkyl group is characterized by the formula CₙH₂₊₁ in which n is at least 3 and less than 20 more particularly, where n=3-16, more particularly where n=3-12, more particularly where n=3-8. A limitation on the length of the alkyl ether resides in the solubility of the compound in solvents suitable for delivery of the compound to a subject by an appropriate route of delivery selected to achieve either acute or chronic administration. Examples of solvents are provided below. The alkyl ether modification may further include cyclical alkyl ethers including cyclohexyl and cyclopentyl derivatives.

A method for making alkyl ethers of polycyclic compounds having a terminal phenol group is provided in Example 1. In this example, the hydroxyl group on the terminal phenol is protected when the compound is reacted with an alkylating agent by a protecting group. The protecting group is subsequently removed. The alkylating agent may be selected from a group consisting of an alkyl halide, a dialkyl sulfate and an alkyl tosylate. The phenolic-OH may be treated with a base resistant protecting group such as tert-butyl, methoxymethyl and 9-anthrylmethyl. The protecting group may be removed by acid hydrolysis, catalytic hydrogenolysis where the hydrogenolysis may include CF₃COOH or by catalytic transfer hydrogenation which may use ammonium formate. The strong base of the method may include sodium hydride.

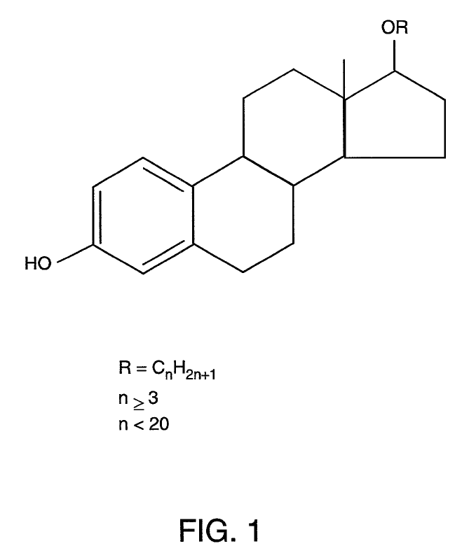

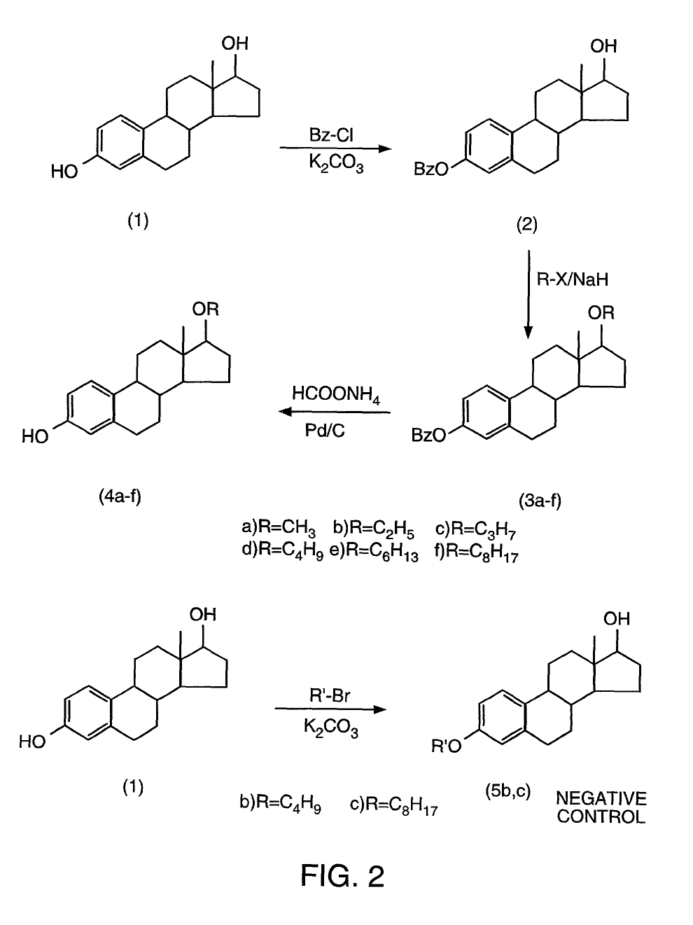

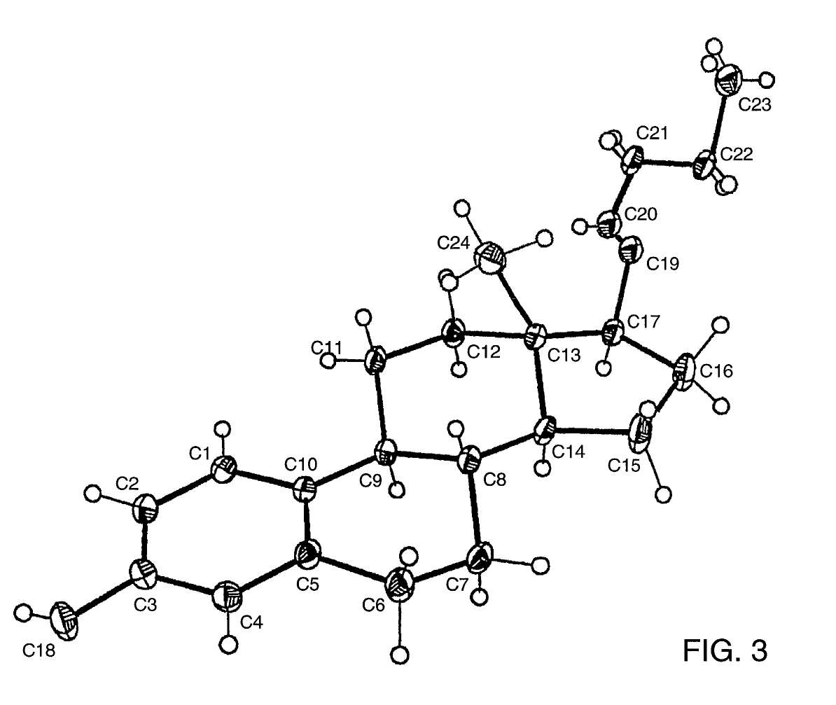

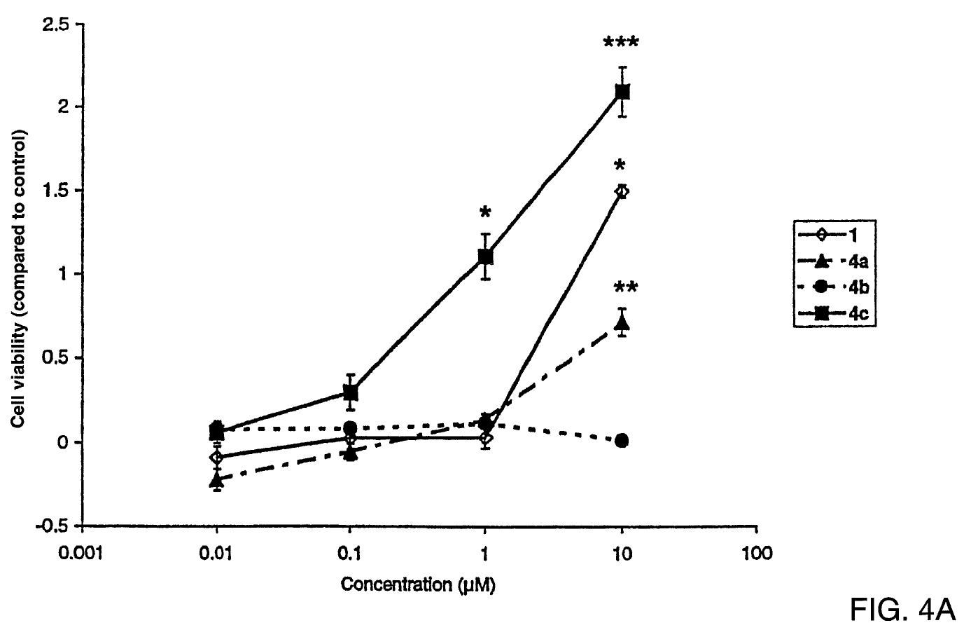

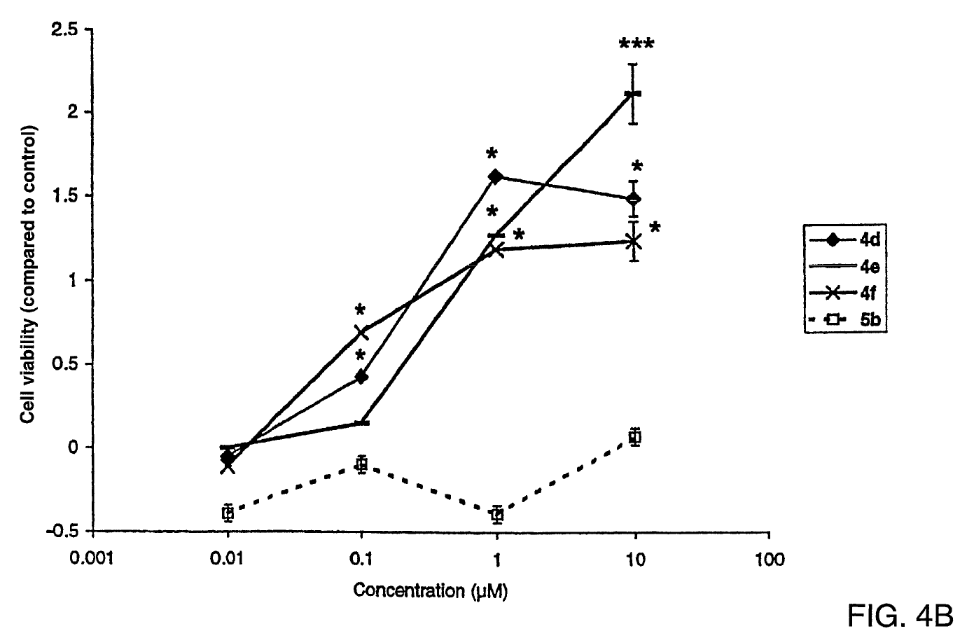

In an embodiment of the invention, an alkyl ether substituted 17-βestradiol is shown schematically in FIG. 1. In addition, the synthetic pathway for making 17-alkyl ether of estradiol is shown in FIG. 2 with a crystallographic structure of 17-O-butylated 17-βestradiol in FIG. 3. The cytoprotection provided by alkyl ether compounds as described has been demonstrated in HT22 assays. (FIG. 4) The observed cytoprotective effect is independent of estrogenic normal activity. Cytoprotective activity using these compounds is not limited to HT22 cells but is applicable to different cell populations and tissues found in a subject and present in vivo and in vitro regardless of whether those cells carried an estrogen receptor or not.

The experimental models for measuring cytoprotection have become established using a range of cell cultures such as HT22, (described below in the Example 2) SK-N-SH (American Type Culture Collection, Rockville, Md.) described in U.S. Pat. No. 5,554,601, erythrocytes and muscle cells and in in vivo animal models. Experimental animals such as rats have been described in which a traumatic event such as ovariectomy itself or additional insult such as an arterial occlusion is generated in ovariectomized and non-ovariectomized animals. (U.S. Pat. Nos. 5,554,601, and 5,859,001). The treated and non-treated rats are then measured for the cytoprotective effect afforded by a range of doses of the compound administered to the animal subject.

The cytoprotective compounds described herein can be used in effective doses to treat patients with acute or chronic degenerative disorders. Examples of acute degenerative disorders include: tissue ischemic events (U.S. Pat. No. 5,877,169, herein incorporated by reference), for example, cerebrovascular disease, subarachnoid hemorrhage or trauma, prevention of ischemia reperfusion injury, prevention of ischemia reperfusion injury in the setting of resuscitation from hypovolemic shock, renal ischemia, myocardial infarction, angina and cardiac ischemia, endothelial inflammation, and cardiotoxicity associated with administration of anti-cancer compositions. Similarly, effective doses of the cytoprotective compounds may be beneficial in treating osteoporosis. (U.S. Pat. No. 5,843,934 herein incorporated by reference). Moreover, the compounds may be used to protect cells in graft tissue during transplantation. (U.S. Pat. Nos. 5,824,672 and 6,207,658 herein incorporated by reference) The compounds may be used to protect aging skin and skin damaged by cytotoxic events either in a cosmetic formulation or as a therapeutic agent. The compounds may be used to protect against vascular degeneration associated with diabetes.

Graft cells include those cells, tissues or organs obtained from a donor by transplantation into a recipient, where the graft cells may be derived from human subjects or from animals and may be transplanted from one subject back into the same subject or from one subject (the donor) into another subject (the recipient) for improving the health of the recipient. In these situations, the donor subject can be a living subject, fetus or a recently deceased subject. The grafts cells and tissues include stem cells, blood cells, bone marrow cells, placental cells, sperm and ova and may further include heart, lungs, corneal tissue or fetal tissue. Accordingly, the compounds described herein may be beneficial in protecting graft cells from damage resulting from oxidative stress.

The cytoprotective compounds described herein can be used to protect neurons from severe degeneration and is an important aspect of treatment for patients with acute or chronic neurodegenerative disorders. Examples of chronic disease include Alzheimer's disease. (U.S. Pat. No. 5,554,601 herein incorporated by reference), Parkinson's disease, Huntingdon's disease, AIDS dementia, Wernicke-Korsakoff's related dementia (alcohol induced dementia), age related dementia, age associated memory impairment, brain cell loss due to any of the following: head trauma, stroke, myocardial infarction, hypoglycemia, ischemia, anoxia, hyopoxia, cerebral edema, arteriosclerosis, diabetic neuropathy, hematoma and epilepsy, spinal cord cell loss due to any of the conditions listed under brain cell loss; and peripheral neuropathy.

Other examples of degenerative diseases, disorders and conditions that may be treatable by a cytoprotective agent include: various bone disorders including osteoporosis, osteomyelitis, ischemic bone disease, fibrous dysplasia, rickets, Cushing's syndrome and osteoarthritis, other types of arthritis and conditions of connective tissue and cartilage degeneration including rheumatoid, psoriatic and infectious arthritis, various infectious diseases, muscle wasting disorders such as muscular dystrophy, skin disorders such as dermatitis, eczema, psoriasis and skin aging, degenerative disorders of the eye including macular degeneration and retinal degeneration, disorder of the ear such as otosclerosis, impaired wound healing, various diseases and conditions of the heart including cardiac ischemia, myocardial infarction, chronic or acute heart failure, cardiac dysrhymias, artrial fibrillation, paroxymial tachycardia, ventricular fibrillation and congestive heart failure, circulatory disorders including atherosclerosis, arterial sclerosis and peripheral vascular disease, diabetes (Type I or Type II), various diseases of the lung including pneumonia, chronic obstructive lung disease (bronchitis, emphysemia, asthma), disorders of the gastrointestinal tract such as ulcers and hernia, dental conditions such as periodontitis, liver diseases including hepatitis and cirrhosis, pancreatic ailments including acute pancreatitis, kidney diseases such as acute renal failure and glomerulonepritis, various blood disorders such as vascular amyloidosis, anerysms, anemia, hemorrage, sickle cell anemia, autoimmune disease, red blood cell fragmentation syndrome, neutropenia, leukopenia, bone marrow aphasia, pancytopenia, thrombocytopenia, hemophilia. The preceding list of diseases and conditions which are potentially treatable with a cytoprotective agent is not intended to be exhaustive or limiting but presented as examples of such degenerative diseases and conditions.

The present compositions may be used for protecting cells including any of the below listed cells or tissues and for treatment of disorders including any of the aforementioned degenerative conditions. Examples of cells that may be protected by the compounds include: stem cells, blood cells, epithelial cells, stromal cells including connective tissue cells, neuronal cells, muscle tissue cells, endocrine tissue cells, whole organ cells, bone cells, skin cells, eye cells, reproductive tract cells and urinary tract cells and tissues that include more than one cell type. Tissues that are protected by the method of the invention may be derived from children, adult or fetal tissue and include, but are not limited to blood and all of its components, including erythrocytes, leukocytes, platelets, serum, central nervous tissue, including brain and spinal cord tissue, neurons, and glia; peripheral nervous tissue, including ganglia, posterior pituitary gland, adrenal medulla, and pineal; connective tissue, including skin, ligaments, tendons, and fibroblasts; muscle tissue, including skeletal, smooth and cardiac tissues or the cells therefrom; endocrine tissue, including anterior pituitary gland, thyroid gland, parathyroid gland, adrenal cortex, pancreas and its subparts, testes, ovaries, placenta, and the endocrine cells that are a part of each of these tissues; blood vessels, including arteries, veins, capillaries and the cells from these vessels; lung tissue; heart tissue and whole organ; heart valves; liver; kidney; intestines; bone, including osteocytes, osteoblasts and osteoclasts; immune tissue, including blood cells, bone marrow and spleen; eyes and their parts; reproductive tract tissues; or urinary tract tissue.

The present compounds may be administered to a subject orally, topically, transdermally through skin or via the mucosal membrane for example the nasal mucosa and buccal mucosa, or parenterally including intravenous, intramuscular and subcutaneous administration. The compound may be further administered subcutaneously using an oil delivery vehicle for improved uptake and sustained effectiveness. Depending on the intended mode, the compositions may be in the form of solid, semi-solid or liquid dosage forms such as for example, tablets, suppositories, pills, capsules, powders, liquids, suspensions, patches, creams, gels, or the like preferably in unit dosage forms suitable for single administration of precise dosages.

The present compositions can be formulated using suitable solvents including cyclodextrin, various proteins, oils such as, corn oil or sesame oil, or alcohols, the solvents of choice being dependent on the route of administration and the need for sustained delivery. For example, intravenous administration of the composition would utilize an aqueous solvent, whereas subcutaneous delivery of the composition might utilize an oil solvent. The therapeutic formulations will include a conventional pharmaceutical carrier or excipient and a therapeutically effective amount of the active agent (cytoprotective compound) and in addition, may include for example, other therapeutic agents, carriers, adjuvants.

The amount of active compound administered will depend on the human or animal subject being treated, the severity of the condition, the manner of administration and the judgement of the prescribing clinician.

Typical compositions contain approximately 0.01-95% by weight of active ingredient with the balance one or more acceptable non-toxic carriers. The percentage of active ingredient will depend upon the dosage form and the mode of administration. Standard formulations have been enumerated in U.S. Pat. No. 6,020,510 (incorporated by reference) and are similarly applicable herein. An effective dose of the active agent as measured in the plasma of a subject may be for example in the range of 5 pg/ml-5000 pg/ml.

All references recited herein are incorporated by reference. The following examples are presented to further illustrate embodiments of the invention but are not intended to be limiting.

### EXAMPLES

### Example 1

### Method of Synthesis of a 17-alkyl Ether of 17β-estradiol

We selectively (and reversibly) protected the 3-OH before alkylating on the 17 position of 17β-estradiol under strong basic condition with the relevant alkyl halide. because alkylation on the phenolic 3-hydroxyl group proceeds under much milder condition than that of the 17 position. Protection of the 3-OH of 17β-estradiol (1) as benzyl (Bz) ether (2) (Qian et al. (1988) J. Steroid Biochem, Vol. 29, pp. 657-664) was achieved by elaboration of the 17β-OH to the corresponding 17β-alkoxyl congeners (3a-f). The 17β-OH group was successfully alkylated with the corresponding alkyl halide in the presence of sodium hydride in dimethylformamide. The 3-benzyl protecting group was removed rapidly under ambient conditions by catalytic transfer hydrogenation using ammonium formate resulting in the desired products (4a-f). (Anwer, et al. (1980) Synthesis, pp. 929-932; Elamin, et al. (1979) J. Org. Chem., Vol. 44, pp. 3442-3444). 3-O-Butyl and octyl ethers of 1(5b,c; Scheme 1) as controls were prepared directly from (1) by using alkyl halide in the presence of potassium carbonate. (The number in parenthesis refer to those in FIG. 2.)

In addition to NMR, mass spectrometry, chromatographic and combustion analyses to characterize the compounds prepared, crystallography data were obtained for two representative 17β-ethers (methoxy and butoxy groups). Summary data for 4d is provided in Table 1. The solid-state conformation (ORTEP-type plot) of 4d is shown in FIG. 3. The crystals were monoclinic and belonged to the P2 (1) space group, and confirmed that the 17-methoxy and butoxy groups assumed β-orientation in the D-ring.

Instruments and Materials

All solvents and material were obtained from FisherScientific (Atlanta, Ga.) or from Aldrich (Milwaukee, Wis.). Estradiol (1) and 3-O methyl-17β-estradiol (5a) were purchased from Sigma (St. Louis, Mo.). Sodium hydride was used as a 60% dispersion in mineral oil. Melting points were determined on a Fisher-Johns melting point apparatus. Thin layer chromatography (TLC) was done on Whatman silica gel plates (on aluminum backing) containing UV fluorescence indicator. All chromatographic purifications were done on gravity columns with 230-435 mesh neutral silica gel using ethyl acetate: hexane 1:4 (v/v) eluent. Elemental analyses were performed by the Atlantic Microlab, Inc. (Norcross, Ga.). NMR spectral data were recorded for all compounds using a Varian XL-300 spectrometer using TMS as internal standard. Mass spectral data were obtained by using atmospheric-pressure chemical ionization (APCI) on a quadrupole ion trap instrument (LCQ, Finnigan MAT, San Jose, Calif.). Analytical reversed-phase high-performance liquid chromatography was performed on a Thermo Separation/SpectraPhysics (Fremont, Calif.) system consisting of an SP8810 isocratic pump, a Rheodyne (Cotati, Calif.) Model 7125 injector valve equipped with a 20-μl sample loop, an SP8450 variable wavelength UV/VIS detector operated at 280 nm, and an SP4290 computing integrator. A 15 cm×4.6 mm id. octadecylsilica column (Phase Sep S5 ODS2, Queensferry, Clwyd, UK) and a mobile phase of acetonitrile containing 1% acetic acid at a flow rate of 1.0 mL/min were used for the analyses.

X-ray crystallography data were collected at 173 K on a Siemens SMART PLATFORM equipped with A CCD area detector and a graphite monochromator utilizing MoKα radiation (1=0.71073 Å). Cell parameters for each structure were refined using up to 8192 reflections and a hemisphere of data (1381 frames) was collected using the w-scan method (0.3° frame width). The first 50 frames were remeasured at the end of data collection to monitor instrument and crystal stability (maximum correction on I was <1%). Absorption corrections by integration were applied based on measured indexed crystal faces. Both structures were solved by the Direct Methods in SHELXTL5, (Sheldrick, G. M. (1998). SHELXTL5. Bruker-AXS, Madison, Wis., USA) and refined using full-matrix least squares. The non-H atoms were treated anisotropically, whereas the hydrogen atoms were calculated in ideal positions and were riding on their respective carbon atoms, except the hydroxyl protons H₁₈ in 4a and H₁₈ and H₂₆ in 4d. These protons were obtained from a Difference Fourier map and refined without any constraints. While no solvent crystallized with 4a, a methanol molecule was found in general position in the lattice of 4d. A total of 196 parameters of 4a were refined in the final cycle of refinement using 2961 reflections with I>2σ(I) to yield R₁ and wR₂ of 5.03% and 12.66%, respectively. For 4d, a total of 247 parameters were refined in the final cycle of refinement using 3294 reflections with I>2σ(I) to yield R₁ and wR₂ of 3.71% and 8.90%, respectively. Refinement was done using F₂. Tables of geometric data, indicating H-bonding interactions are provided here for one compound and are further available on the Cambridge Data base for crystallography. (Steps in the synthetic pathway shown in FIG. 2.)

3-Benzyloxyestra-1,3,5(10)-trien-173-ol (2)

(Quian et al. (1988) J. Steroid Biochem, Vol. 29, pp. 657-664). Benzyl bromide was added to 5 g (18 mmol) of 1 and 10 g (72 mmol) potassium carbonate in 100 ml of acetone 5.7 g (4.0 mL, 34 mmol). The mixture was refluxed overnight. Upon cooling the solid was removed by filtration. The filtrate was collected and acetone was removed in vacuo leaving behind clear yellowish oil, which solidified on standing. Recrystallization from ethyl acetate/hexane gave 6.1 g (93% yield) of a white fluffy solid, m.p. 119-121° C.; TLC R_{f} 0.23; ¹H-NMR (CDCl₃) δ: 7.44-7.19 (m, 5H); 6.78(dd, J=8.7 Hz and J=2.7 Hz, 1H); 6.72 (d, J=2.4 Hz, IH); 5.05 (s, 3H); 3.37 (tr, J=8.4 HZ, 1H); 2.87-2.82 (m, 2H); 2.34-1.18 (m, H); 0.78 (s, 3H). MS: m/z 363 [M+H]⁺.

General Procedure for the Preparation of 3-Benzyloxy-17β-alkoxyestra-1,3,5(10)-triene (3a-f)

Compound 2 (2) (0.8 g, 2.2 mmol) was dissolved in 5 ml anhydrous DMF and, then, sodium hydride (0.3 g) was added. The mixture was stirred at room temperature for 30 min before the addition of 20 mmol alkyl-halide. The stirring was continued overnight. The reaction mixture was quenched by pouring it into 20 mL of dilute hydrochloric acid and extracted with methylene chloride. The organic phase was dried over Na₂SO₄ and the solvent removed in vacuo leaving behind a clear, yellowish oil which solidified on standing. The crude products were purified by either recrystallization or column chromatography.

3-Benzyloxy-17β-methoxyestra-1,3,5(10)-triene (3a)

Recrystallization from methanol, 63% yield. Yellowish solid, m.p. 92-94° C.; TLC R_{f} 0.83;¹H-NMR (CDCl₃) δ: 7.32-7.48 (m, 5H), 7.22 (dd, J=8.7 and J=2.10 Hz, 1H), 6.80 (d, J=2.4, 1H), 5.05 (s, 2H), 3.39 (s,3H), 3.33 (t; 1H, J=8.7), 2.83 (m, 2H), 1.22-2.34 (m, 13H), 0.80 (s, 3H). MS: m/z 377 [M+H]⁺.

3-Benzyloxy-17β-ethoxyyestra-1,3,5(10)-triene (3b)

Column chromatography, 49% yield. TLC R_{f} 0.71; ¹H-NMR (CDCl₃) δ: 7.45-7.30 (m, 5H), 6.79 (dd, J=8.7 and J=2.10 Hz, 1H), 6.71 (d, J=2.5, 1H), 5.02 (s, 2H), 3.55 (dq, J=6.9 Hz and 2.1 Hz, 1H), 3.48 (dq, J=7.0 Hz and 2.1 Hz, 1H), 3.39 (t, J=8.1 Hz, 1H), 2.84-2.81 (m, 2H), 2.31-1.37 (m,13H), 1.18 (t, J=6.9 Hz, 3H), 0.79 (s, 3H). MS: m/z 391 [M+H]⁺.

3-Benzy [oxy-175-propoxyestra-1,3,5(10)-triene (3c)

Column chromatography. Yield. 54%. White solid. TLC R_{f} 0.68, ¹H-NMR (CDCl₃) δ: 7.44-7.37 (m, 5H), 6.75 (dd, J=8.6 and J=2.1 Hz, 1H), 6.70 (d, J=2.7, 1H), 5.02 (s, 2H), 3.41 (dt, J=6.9 Hz and 2.4 Hz, 2H), 3.37 (t, J=8.4 Hz, 1H), 2.84-2.81 (m, 2H), 2.31-1.37 (m, 15), 0.92 (t, J=6.6 Hz, 3H), 0.79 (s,3H). MS: m/z 405 [M+H]⁺.

3-Benzyloxy-170-butoxyestra-1,3,5(10)-triene (3d)

Column chromatography, yield 52%. White solid. TLC R_{f} 0.65, ¹H-NMR (CDCl₃) δ: 7.45-7.30 (m, 5H), 6.79 (dd, J=8.7 and J=2.10 Hz, 1H), 6.71 (d, J=2.5, 1H), 5.02 (s, 2H), 3.55 (dq, J=6.9 Hz and 2.1 Hz, 1H),3.48 (dq, J=7.0 Hz and 2.1 Hz, 1H), 3.39 (t, J=8.1 Hz, 1H), 2.84-2.81 (m, 2H), 2.31-1.37 (m, 13H), 1.18 (t, J=6.9 Hz, 3H), 0.79 (s, 3H). MS: m/z 419 [M+H]⁺.

3-Benzyloxy-17β-hexyloxyestra-1,3,5(10)-triene (3e)

Column chromatography, yield 63%. White solid. TLC R_{f} 0.75, ¹H-NMR (CDCl₃) δ: 7.49-7.34 (m, 5H), 6.74 (dd, J=8.7 and J=2.7 Hz, 1H), 6.71 (d, J=2.7, 1H), 4.98 (s, 2H), 3.44 (dt, J=7.6 Hz and 2.7 Hz, 2H) 3.36 (t, J=8.1 Hz, 1H), 3.55 (dq, J=6.9 Hz and 2.1 Hz, 1H), 3.48 (dq, J=7.0 Hz and 2.1 Hz, 1H), 3.39 (t, J=8.1 Hz, 1H), 2.84-2.81 (m, 2H), 2.31-1.37 (m, 13H), 1.18 (t, J=6.9 Hz, 3H), 0.79 (s, 3H). MS: m/z 447 [M+H]⁺.

3-Benzyloxy-17β-actyloxyestra-1,3,5(10)-triene (3f)

Column chromatography, 55% yield, yellow oil. TLC R_{f} 0.85, ¹H-NUR (CDCl₃) δ: 7.45-7.30 (m, 5H), 6.79 (dd, J=8.7 and J=2.10 Hz, 1H), 6.71 (1H, J=7.7), 5.02 (s, 2H), 3.55 (dq, J=6.9 Hz and 2.1 Hz, 1H), 3.48 (dq, J=7.0 Hz and 2.1 Hz, 1H), 3.39 (t, J=8.1 Hz, 1H), 2.84-2.81 (m, 2H), 2.31-1.37 (m, 13H), 1.18 (t, J=6.9 Hz, 3H), 0.79 (s, 3H). MS: m/z 475 [M+H]⁺.

General Procedure for the Preparation of 17β-alkoxyestra-1,3,5(10)-triene (4a-f)

To a solution of 2.0 mmol 3a-f in 10 mL of methanol was added 0.2 g of Pd/C (10%) and ammonium formate (1.00 g, 16mmol). The reaction mixture was stirred at room temperature for 1 hr. Then the Pd/C was then removed by filtration and solvent was removed in vacuo. To the oily residue water was added and the resulting solid was collected by filtration. Either recrystallization or column chromatography was used for purification.

17β-Methoxyestra-1,3,5(10)-trien-3-ol (4a)

Recrystallization from methanol, 50% yield. White solid, m.p. 242-244° C.; TLC: R_{f} 0.48; ¹H-NMR (DMSO) δ: 7.05 (d, J=8.40 Hz, 1H), 6.51(dd, J=8.40 Hz and 2.10 Hz, 1H), 6.45 (d, J=2.40 Hz, 1H), 3.30 (s, 3H), 3.28 (t, j=8.25 Hz, 1H); 2.73-2.72 (m, 3H); 2.56-2.50 (m, 1H); 2.30-1.22 (m, 13H); 0.74 (s, 3H). ¹³C-NMR (DMSO) δ: 156.7, 139.3, 132.7, 128.0, 116.8, 114.5, 92.2, 58.7, 51.7, 45.6, 44.6, 40.2, 39.8, 31.1, 29.2, 28.8, 28.1, 24.4, 13.6; MS: m/z 287 [M+H]⁺, 255 [M-OCH₃]⁺. Anal. C, H.

17β-Ethoxyestra-1,3,5(10)-trien-3-ol (4b)

Recrystallization from methanol, 50% yield, white solid; TLC: R_{f} 0.57; ¹H-NMR (CDCl₃) δ: 7.08 (d, J=8.7 Hz, 1H), 6.55 (dd, J=8.4 Hz, 2.1 Hz, 1H), 6.48 (d, J=2.4 Hz, 1H), 3.65 (qd, J=7.02 Hz and 2.48 Hz, 1H), 3.56 (qd, J=7.05 Hz and 2.48 Hz, 1H), 3.44 (t, J=8.4 Hz, 1H), 2.76-2.72 (m, 2H), 2.20-1.10 (m, 13H), 1.20 (t, J=7.2 Hz, 3H), 0.80 (s, 3H); ¹³C-NMR (CDCl₃) δ: 155.72, 138.83, 132.64, 127.19, 116.1, 113.7, 89.8, 66.1, 50.8, 44.5, 43.8, 39.3, 38.6, 30.1, 28.6, 27.8, 27.01, 23.5, 15.8 11.9; MS: m/z 301 [M+H]⁺, 255 [M-OC₂H₅]⁺. Anal. C, H.

17β-Propoxyestra-1,3,5(10)-trien-3-ol (4c)

Recrystallization from methanol, 50% yield, white solid; TLC: R_{f} 0.54; ¹H-NMR (CDCl₃) δ: 7.08 (d, J=8.7 Hz, 1H), 6.55 (dd, J=8.4 Hz, 2.1 Hz, 1H), 6.48 (d, J=2.4 Hz, 1H), 3.45 (dt, J=6.77 Hz and 1.67 Hz, 2H), 3.31 (m, 3H), 2.76-2.72 (m, 2H), 2.20-1.10 (m, 13H), 0.94 (td, J=7.2 Hz and 1.92 Hz, 3H), 0.72 (s, 3H); ¹³C-NMR (CHCl₃) δ: 154.0, 137.9, 131.7, 126.2, 115.0, 112.5, 89.0, 71.9, 50.1, 43.8, 43.2, 38.5, 38.0, 29.5, 27.9, 27.1, 26.3, 23.1, 22.9, 11.4, 10.4; MS: m/z 315 [M+H]⁺, 255 [M-OC₃H₇]⁺. Anal. C, H.

17β-Butoxyestra-1,3,5(10)-trien-3-ol (4d)

Recrystallization from methanol, 50% yield, white solid, m.p. 77-81° C.; TLC: R_{f} 0.47; ¹H-NMR (CDCl₃) δ: 7.08 (d, J=8.7 Hz, 1H), 6.55 (dd, J=8.4 Hz, 2.1 Hz, 1H), 6.48 (d, J=2.4 Hz, 1H), 3.50 (dqn, J=7.00 Hz and 2.01 Hz, 1H), 3.45 (dqn, J=7.11 Hz and 1.85 Hz, IH), 3.31 (t, J=8.4 Hz, 1H), 2.76-2.72 (m, 2H), 2.20-1.10 (m, 17H), 0.85 (t, J=7.2 Hz, 3H), 0.72 (s, 3H); ¹³C-NMR (CHCl₃) δ: 6:153.3, 138.3, 132.7, 126.5, 115.2, 112.5, 89.1, 70.0, 50.3, 43.9, 43.3, 38.6, 38.1, 32.3, 29.6, 28.2, 27.1, 26.5, 23.0, 19.4, 14.0, 11.6; MS: m/z 329 [M+H]⁺, 255 [M-OC₄H₉]⁺.

17β-Hexyloxyestra-1,3,5(10)-trien-3-ol (4e)

Column chromatography, 70% yield, white semisolid. TLC: R_{f} 0.47; ¹H-NMR (CDCl₃) δ: 7.12 (d, J=8.4 Hz, 1H), 6.62 (dd, J=8.3 Hz, 2.7 Hz, 1H), 6.54 (d, J=2.5 Hz, 1H), 3.43 (dt, J=7.6 Hz and 2.7 Hz, 2H) 3.36 (t, J=8.1 Hz, 1H), 2.80-2.77 (m, 2H), 2.25-1.25 (m, 18H), 0.89-0.85 (m, 6H), 0.78 (s, 3H); ¹³C-NMR (CHCl₃) δ: 153.2, 138.2, 132.6, 126.4, 115.1, 112.5, 89.0, 70.3, 50.2, 43.8, 43.3, 38.5, 38.0, 31.6, 30.1, 29.5, 28.1, 26.5, 25.8, 23.0, 22.6, 14.0, 11.6; MS: m/z 357 [M+H]⁺, 255 [M-OC6H₁₃]⁺. Anal. C, H.

17β-Octyloxyestra-1,3,5(10)-trien-3-ol (4f)

Column chromatography, 75% yield, pale yellow semi-solid. TLC: R_{f} 0.50; ¹H-NMR (CDCl₃) δ: 7.12 (d, J=8.7 Hz, 1H), 6.62 (dd, J=8.4 Hz, 2.2 Hz, 1H), 6.53 (d, J=2.3 Hz, 1H), 3.49 (qd, J=6.79 Hz and 2.52 Hz, 1H), 4.31 (qd, J=6.72 Hz and 2.55 Hz, 1H), 3.37 (t, J=8.5 Hz, 1H), 2.81-2.76 (m, 2H), 2.22-1.18 (m, 22H), 0.87-0.83 (m, 6H), 0.79 (s, 3H);¹³C-NMR (CHCl₃) δ: 153.3, 138.2, 132.6, 126.5, 115.2, 112.6, 89.1, 70.3, 50.2, 43.9, 43.3, 38.6, 38.0, 31.8, 30.1, 29.7, 29.4, 29.3, 28.1, 27.1, 26.4, 26.2, 23.0, 22.6, 14.0, 11.6; MS: m/z 385 [M+H]⁺, 255 [M-OC₈H₁₇]⁺. Anal. C, H.

General Procedure for the Preparation of 3-Alkoxyestra-1,3,5(10)-triene (5b,c)

To compound 1 (0.5 g, 1.8 mmol) and potassium carbonate (1.00 g, 7.2.mmol) in 5 ml of acetone 10 mmol of 1-bromobutane or 1-bromooctane was added. The mixture was refluxed overnight then allowed to cool down and was filtered. The acetone was removed and the oily residue was purified.

3-Butoxyestra-1,3,5(10)-trien-17β-ol (5b)

Recrystallization from methanol: water 1:1 (v/v), 68% yield. White solid; m.p. 86-88° C.; TLC R_{f} 0.62; ¹H-NMR (CDCl₃) δ: 7.17 (d, J=8.7 Hz, 1H), 6.70 (dd, J=8.4 Hz and 2.40 HZ, 1H), 6.62 (d, J=2.4 Hz, 1H), 3.93 (t, J=6.30 Hz, 2H), 3.71 (t, J=8.1 Hz, 1H), 2.86-2.80 (m, 2H), 2.20-1.10 (m, 17H), 0.96 (t, J=7.2 Hz, 3H), 0.77 (s, 3H); ¹³C-NMR (CHCl₃) δ: 156.9, 137.7, 132.3, 126.1, 114.4, 111.9, 81.7, 67.5, 49.9, 43.8, 43.1, 38.7, 36.6, 31.3, 30.4, 29.7, 27.2, 26.3, 23.0, 19.2, 13.7, 10.9. MS: m/z 311 [M-OH]⁺.

3-Octyloxyestra-1,3,5(10)-trien-17β-ol (5c)

Column chromatography, 72% yield. White solid, m.p. 64-66° C.; TLC R_{f} 0.70;¹H-NMR (CDCl₃) δ: 7.18 (d, J=8.7 Hz, 1H), 6.7(dd, J=8.7 Hz and 2.7 Hz, 1H), 6.62 (d, J=2.8 Hz, 114), 3.91 (t, J=6.6 Hz, 2H), 3.73 (t, J=8.4 Hz, 1H), 2.85-2.82 (m, 2H), 2.20-1.10 (m, 25 H), 0.88 (t, J=6.6 Hz, 3H), 0.77 (s, 3H); ¹³C-NMR (CHCl₃) δ: 156.9, 137.8, 132.4, 126.2, 114.5, 112.0, 81.9, 70.3, 67.9, 50.0, 43.9, 43.2, 38.8, 38.1, 36.6, 30.1, 29.7, 29.4, 29.2, 27.2, 26.4, 26.2, 23.1, 22.6, 14.0, 11.0. MS: m/z 368 [M-OH]⁺. Anal. C, H.

TABLE 1
Crystal data and structure refinement for 4d.

| Identification code | 4d | |
|---|---|---|
| Empirical formula | C23 H36 O3 | |
| Formula weight | 360.52 | |
| Temperature | 173(2) K | |
| Wavelength | 0.71073 Å | |
| Crystal system | Monoclinic | |
| Space group | P2(1) | |
| Unit cell dimensions | a = 8.6418(4) Å | α = 90°. |
| | b = 9.5698(5) Å | β = 102.021(1)°. |
| | c = 12.8534(7) Å | γ = 90°. |
| Volume | 1039.67(9) Å³ | |
| Z | 2 | |
| Density (calculated) | 1.152 Mg/m³ | |
| Absorption coefficient | 0.074 mm⁻¹ | |
| F (000) | 396 | |
| Crystal size | 0.21 × 0.21 × .13 mm³ | |
| Theta range for data collection | 1.62 to 27.50°. | |
| Index ranges | −11 · h · 11, −12 · k · 8, −16 · 1 · 16 | |
| Reflections collected | 7032 | |
| Independent reflections | 3784 [R (int) = 0.0233] | |
| Completeness to theta = 27.49° | 99.8% | |
| Absorption correction | Integration | |
| Max. and min. transmission | 0.996 and 0.987 | |
| Refinement method | Full-matrix least-squares on F² | |
| Data/restraints/parameters | 3784/1/247 | |
| Goodness-of-fit on F² | 0.976 | |
| Final R indices [I > 2sigma(I)] | R1 = 0.0371, wR2 = 0.0890 [3294] | |
| R indices (all data) | R1 = 0.0434, wR2 = 0.0917 | |
| Absolute structure parameter | −0.6(10) | |
| Extinction coefficient | 0.007(2) | |
| Largest diff. peak and hole | 0.205 and −0.172 e.Å⁻³ | |

R1 = Σ(11Fₒ1-1F_{c}11)/Σ1Fₒ1
wR2 = [Σ[w(Fₒ²-F_{c}²)²]/Σ[w(Fₒ²)²]]^{1/2}
S = [Σ[w(Fₒ²-F_{c}²)²]/(n-P)]^{1/2}w-- 1/[o²(Fₒ²) + (0.0370*p)² + 0.31*p], p = [max(Fₒ²,0) + 2*; F_{c}²]/3

### Example 2

### Biological Activity of Compounds

Cytotoxicity Studies

Mouse clonal hippocampal HT-22 cells were cultured in DMEM media supplemented with 10% fetal bovine serum under standard cell culture conditions. All wells in the 96 well culture plate contained approximately 5,000 HT-22 cells as determined by a Neubauer hemacytometer and the cells were incubated for 24 hrs before the compounds were added. The estradiol derivatives were purified recrystallization or column chromatography and were free from (1) as determined by HPLC. All agents were dissolved in absolute ethanol and diluted, with the culture media, to a final concentration of 0.01 μM; 0.1 μM; 1.0 μM; and 10 μM in their respective wells. The cells were further incubated for 24 hrs before sodium glutamate in a solution of phosphate buffer was added. Cell viability was quantified 2 hrs later by the calcein AM assay (Green, P. S., E. J.Perez, T. Calloway and J. W. Simpkins: (2000), Journal of Neurocytology, Vol. 29, pp. 419-423) in a phosphate buffer solution.

Statistical Analysis

ANOVA was used to determine the significance of differences among groups. Comparison between groups were done using the Tukey test. Ap<0.05 was considered significant. The results are shown in FIG. 4.

Compared to (1), 4c-f of the six 17β-O-alkylestradiols showed improved neuroprotection in a dose-dependent manner against the glutamate-induced oxidative damage in murine HT-22 cells at concentrations of 0.1 μM and higher (FIG. 4). These compounds were essentially equipotent at 1 μM (approximately twice as many cells were viable compared to the control), and showed no apparent relationship with a single molecular property such as lipophilicity (based on the calculated log P). The logarithm of the 1-octanol/water partition coefficient (log P) was calculated by an atom fragment method implemented in the molecular modeling package HyperChem version 6.0 (Hypercube, Gainesville, Fla.): Ghose, et al., (1988) J. Comput Chem, Vol. 9, pp. 80-90. The obtained log P values were as follows: 4.01 (1), 4.29 (4a), 4.63 (4b), 5.10 (4c), 5.49 (4d), 7.08 (4f). The calculated log P for the 3-alkylestradiols were 4.09 (5a), 5.25 (5b), and 6.83 (5c).

The butyl (4e) and octyl ether (4f) were neuroprotective to a similar extent at a concentration of 10 μM and 1 μM. In contrast, the parent compound (1) and 17β-methylestradiol were effective only at 10 μM, and were less active then 4c and 4e at this concentration. 17β-ethylestradiol (4a) was ineffective even at 10 μM. The 5(b) and 5c ethers in which the phenolic hydroxyl in the A-ring were blocked were ineffective with respect to cytoprotection.

The complex relationship of cytoprotection and 17-alkoxy chain length was surprising. A comparison of the solid-state conformation of 4a and 4d revealed no apparent differences in the preferred geometry of the steroid backbone between a representative “active” (4e) and an “inactive” (4a) ether derivative of (1). Without wishing to be limited by theories, we propose that a possible explanation for the above described behavior is that the interaction of the alkyl chain of the 17β-substituent with the target site or the lipoidal cell membrane plays an important role in the efficacy of the derivative as a cytoprotectant. Thus, 4a and 4b having a compact alkyl group may not have the flexibility (i.e., sufficient degrees of freedom for bond rotation) to embed into a cell membrane effectively; however, a longer alkyl chain (C≧3) may provide this property.

In summary, 17β and 17α-alkyl ethers of estradiol have dose-dependent cytoprotective effects in vitro. Moreover, this effect is manifested at lower concentration (<1 μM) than that of the parent compound.

### Example 3

### Cytoprotection (Neuroprotection) is Unrelated to Binding to Estrogen Receptor

Human cloned estrogen receptors (ER) for both ERα and ERβ areas were mixed with radiolabeled 17β-estradiol and with no other compound (total binding), with excessive amount of diethylstilbesterol (non-specific binding), or with cold (unlabeled) estradiol, or the test compound. All groups were determined in duplicate or triplicate. 17β-estradiol was tested at concentrations of 0.1, 1 and 10 mM, while all other test compounds were assayed at 10 mM.

17β-estradiol produced a dose-dependent inhibition of binding of the labeled estradiol to both receptors with approximately equal affinity. The activity of 17β estradiol was assigned a value of 1. Test compounds were compared to the binding inhibition produced by 17β-estradiol.

Values of <0.01 indicate no evidence of binding of the test compound to the receptor.

Values of <0.1 indicate weak binding (less than 10% of the activity of 17β-estradiol. ND indicates that the compound has not been tested at this time

TABLE 2
Comparison of compounds based on neuroprotective
properties and estrogen receptor binding.

| | NEURO- | | |
|---|---|---|---|
| | PROTECTION | | |
| | (Effectiveness | ERα BINDING | ERβ BINDING |
| COMPOSITE | relative to E2) | (Relative to E2) | (Relative to E2) |
| 17beta E2 | 1 | 1 | 1 |
| Ent-E2 | 1.14117 | <0.028 | <0.028 |
| 17alpha E2 | 1.35856 | ND | ND |
| 17-ethyl ether | | <0.01 | ND |
| 17-octyl ether | | <0.01 | <0.01 |
| 17-propyl ether | | <0.01 | ND |

Although certain preferred embodiments of the present invention have been described, the spirit and scope of the invention is by no means restricted to what is described above. In addition to the above references incorporated by reference, Prokai et al. (2001) J. Med. Chem. 2001, Vol 44, 110-114 is also incorporated by reference.

What is claimed is:

1. A cytoprotective compound, comprising: a polycyclic compound optionally having two, three or four carbon rings, the compound also having a first end and a second end wherein a phenol group is located at the first end and a terminal carbon ring is located at the second end, the terminal carbon ring having an alkyl ether functional group, the alkyl portion of which having a formula CₙH₂ₙ₊₂ wherein n is at least 3 and less than 20.

2. A cytoprotective compound, according to claim 1, wherein the polycyclic compound is a four ring compound, and the carbon ring at the second end is a D ring, the D ring having the alkyl ether functional group.

3. A cytoprotective compound according to claim 2, wherein the four ring compound is an estrogen compound.

4. A cytoprotective compound according to claim 3, wherein the alkyl ether functional group is on carbon 17 in the D ring.

5. A cytoprotective compound according to claim 4, wherein the alkyl ether functional group is in an orientation selected from the group consisting of an alpha or beta isomeric orientation on the carbon.

6. A cytoprotective compound, according to claim 1, wherein the alkyl group is selected from a long chain saturated-alkyl group, long chain unsaturated alkyl group and a cyclo alkyl group.

7. A cytoprotective compound according to claim 4, comprising: a 17-butoxyestra 1,3,5(10) triene-3-ol.

8. A cytoprotective compound, according to claim 4, comprising: a 17-hexyloxyestra-1,3,5(10)-triene-3-ol.

9. A cytoprotective compound according to claim 4, comprising: a 17-octyloxyestra-1,3,5(10)-triene-3-ol.

10. A cytoprotective compound, comprising: an estrogen compound having a terminal phenol group at a first end of the compound and a carbon ring at a second end of the compound, the carbon ring at the second end having an alkyl ether functional group, the alkyl portion of the group having a formula CₙH₂ₙ₊₂ wherein n is at least 3 and less than 20.

11. A pharmaceutical formulation, comprising: a cytoprotection effective dose of a polycyclic compound having a phenolic ring at a first terminal position, any of one, two or three additional ring structures and an alkyl ether functional group on a carbon ring in a second terminal position.

12. A method of retarding the development of a degenerative condition associated with a population of cells in a subject, comprising:
administering to the subject predisposed to the degenerative condition, an effective amount of a polycyclic phenolic compound in a physiologically acceptable formulation, the polycyclic phenolic compound having a phenol located at a first terminal position, and optionally any of one, two or three additional ring structures; the compound having an alkyl ether group located on a carbon ring at a second terminal position, the alkyl ether group having an alkyl with a formula CₙH₂ₙ₊₂ wherein n is at least 3 and less than 20, the compound retarding the development of the degenerative condition.

13. A method according to claim 12 wherein the polycyclic phenolic compound is a four ring compound and the carbon ring at the second end is a D ring, the D ring having the alkyl ether functional group on the 17 carbon position.

14. A method according to claim 12, wherein the population of cells is selected from cells or tissues comprising any of the group consisting of stem cells, blood cells, epithelial cells, stromal cells including connective tissue cells, neuronal cells, muscle tissue cells, endocrine tissue cells, whole organ cells, bone cells, eye cells, skin cells, reproductive tract cells and urinary tract cells.

15. A method according to claim 12, wherein the condition is a bone disorder.

16. A method according to claim 15, wherein the bone disorder is selected from osteoporosis, osteomyelitis, ischemic bone disease, fibrous dysplasia, rickets, Cushing's syndrome and osteoarthritis.

17. A method according to claim 12, where the condition is a cardiac disorder.

18. A method according to claim 17, wherein the cardiac disorder is selected from cardiac ischemia, myocardial infarction, chronic or acute heart failure, cardiac dysrhymias, atrial fibrillation, paroxymial tachycardia, ventricular fibrillation and congestive heart failure.

19. A method according to claim 12, wherein the condition is selected from a skin disorder, a pulmonary disorder, a hepatic disorder, a renal disorder, a vascular disorder and an autoimmune disorder.

20. A method according to claim 12, wherein the condition is an eye disorder.

21. A method according to claim 20, wherein the eye disorder is selected from the group consisting of macular degeneration and retinal degeneration.

22. A method according to claim 12, wherein the condition is a neurodegenerative disease.

23. A method according to claim 22, wherein the neurodegenerative condition is selected from Alzheimer's disease, Parkinson's disease, Huntingdon's disease, age related dementia, age associated memory impairment, head trauma, stroke, anoxia, hypoxia and cerebral edema and diabetic neuropathy.

24. A method according to claim 23, wherein the condition is an ischemic condition.

25. A method according to claim 24, wherein the ischemic condition is selected from cerebrovascular disease, subarachnoid hemorrhage or trauma, prevention of ischemia reperfusion injury, renal ischemia, myocardial infarction, angina and cardiac ischemia.

26. A method of synthesizing an estrogen compound having a phenolic A ring and an alkyl ether functional group on carbon 17, comprising:
a. protecting —OH on the phenolic A ring;
b. alkylating the 17-OH with an alkylating agent in the presence of a strong base;
c. removing the protecting group from —OH on the phenolic A ring; and
d. purifying the 17-alkyl ether estrogen compound.

27. A method according to claim 26, wherein the —OH on the phenolic A ring is in the carbon 3 position.

28. A method according to claim 26, wherein the alkylating agent is selected from the group consisting of a alkyl halide, a dialkyl sulfate and an alkyl tosylate.

29. A method according to claim 26, further comprises: treating the —OH on the phenolic A ring with a base resistant protecting group.

30. A method according to claim 26, further comprising a protecting group being removable by acid hydrolysis or catalytic hydrogenolysis.

31. A method according to claim 29, wherein the base resistant protecting group is selected from tert-butyl, methoxymethyl, and 9-anthrylmethyl.

32. A method according to claim 30, wherein the protecting group is a benzyl or substituted benzyl group capable of being cleaved by hydrogenolysis.

33. A method according to claim 30, wherein the hydrogenolysis is achieved using CF₃COOH.

34. A method according to claim 26, wherein the strong base is sodium hydride.

35. A method according to claim 26, further comprising: removing the protecting group by catalytic transfer hydrogenation.

36. A method according to claim 35, wherein the catalytic transfer hydrogenation utilizes ammonium formate.

37. A method of treating a subject having a degenerative disorder, comprising: obtaining at least one 17-O-alkyl ether of estrogen in a pharmaceutical formulation; and administering an effective dose of the 17-O-alkyl ether of estrogen to the subject so as to treat the degenerative disorder.

38. A method according to claim 37, wherein the degenerative disorder is a neurodegenerative disorder.

39. A method according to claim 38, wherein the neurodegenerative disorder is Alzheimer's disease and the effective dose of the 17-O-alkyl ether of the estrogen compound provides protection of a population of nerve cells from progressive cell damage leading to cell death otherwise occurring with out the intervention.

40. A method according to claim 37, further comprising administering the effective dose by any of an oral route, transdermal, topical or parenteral route of administration.

41. A method according to claim 37, wherein the degenerative disorder is an ischemia.

42. A method according to claim 41, wherein the ischemic condition includes ischemic reperfusion injury, myocardial infarction and cardiac ischemia.

43. A method of conferring cytoprotection of a population of cells, comprising:
(i) providing an 17β-O-alkyl ether of an estrogen compound; and
(ii) administering the compound in an effective dose to the population of cells so as to confer cytoprotection on the population of cells.

43. A method according to claim 42, wherein the population of cells is in a subject.

44. A method according to claim 42, wherein the population of cells is ex vivo.

45. A method according to claim 42, wherein the population of cells is graft cells.
